(19) United States
(12) Patent Application Publication
(10) Pub. No.: US 2010/0179311 A1
(43) Pub. Date: Jul. 15, 2010

(54) REACTIONS OF GROUP 16 ELEMENTS

(75) Inventors: Martin John Earle, Belfast (GB); Eva Boros, Belfast (GB); Kenneth Richard Seddon, Belfast (GB); Manuela A. Gilea, Belfast (GB); Joseph S. Vyle, Belfast (GB)

Correspondence Address: CARSTENS & CAHOON, LLP, P O BOX 802334, DALLAS, TX, 75380, US

(73) Assignee: THE QUEEN'S UNI IONIC LIQUID LAB RESEARCH CENTRE, Belfast (GB)

(21) Appl. No.: 12/063,764

(22) PCT Filed: Aug. 21, 2006
(86) PCT No.: PCT/GB06/03129

§ 371 Date: Apr. 1, 2008

(30) Foreign Application Priority Data
Aug. 19, 2005 (GB) 0517074.1

(51) Int. Cl. C07F 9/09; C07H 1/00

(52) U.S. Cl. 536/26.1; 568/17; 558/113

(57) ABSTRACT

Reactions of Group 16 elements involving the addition of atoms such as sulfur, selenium or tellurium to organic or inorganic molecules comprising use of an ionic liquid as a reaction medium.

The present invention relates to reactions of Group 16 elements, and more specifically to reactions involving the addition of atoms of sulfur, selenium or tellurium to organic or inorganic molecules.

As used herein, the term “Group 16” refers to Group 16 of the Periodic Table of “Organic Chemistry”, Second Edition, Ed. K. Peter C. Vollhardt & Neil E. Schore, pub. W. H. Freeman & Co., New York.

The introduction of oxygen atoms into organic molecules is readily achievable using a variety of oxidizing agents. However analogous reactions for introducing other Group 16 elements are generally more complicated to carry out and usually involve the use of expensive and unusual reagents. An example is the use of Beaucage Reagent (3H-1,2-benzodithiol-3-one-1,1-dioxide) for carrying out sulfurisation reactions. It would be particularly advantageous to be able to avoid the use of such reagents, and to be able to carry out the desired reactions using such other Group 16 elements in elemental form.

Accordingly, although there are many instances where it is desired to produce Group 16 analogues of oxygen-containing compounds, the production of such analogues has been impeded by the complexity of the synthetic procedures involved. This is especially the case with sulfur and selenium analogues of nucleosides, nucleotides and oligonucleotides, which frequently have applicability in medicine and biological/biochemical research. As used herein, the expression “Group 16 analogues of oxygen-containing compounds” refers to a compound containing one or more atoms of a Group 16 element other than oxygen, in a position in the compound that would normally be occupied by an oxygen atom. Examples are phosphorothioate analogues of phosphatediesters in natural products.

It is also desired to carry out addition reactions in which Group 16 elements other than oxygen are introduced by way of an addition or substitution reaction to an organic compound. In the case of addition of sulfur, such reactions may be termed “sulfurization reactions”.

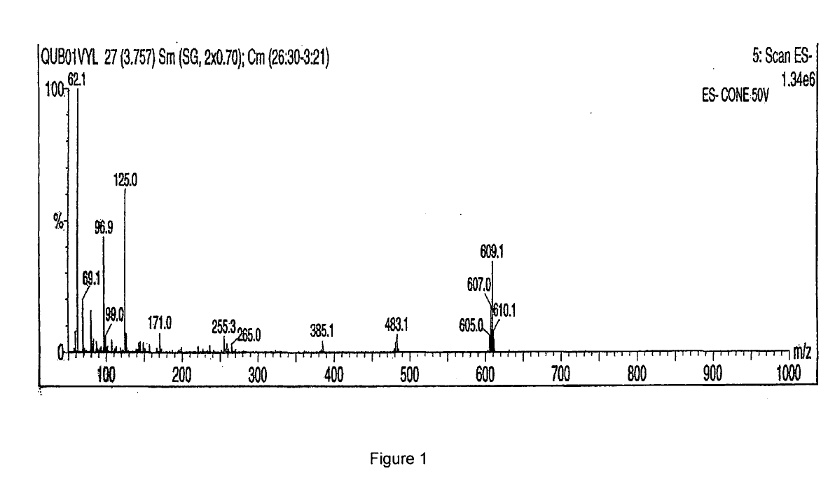

An example of a sulfur-containing oligonucleotide analogue is Vitravene™. In this regard, the first license for a drug based upon a short DNA sequence (or oligonucleotide) was granted in 1998 for the use of this product in the treatment of cytomegalovirus-induced retinitis. By the end of 2004 there were over 40 pharmaceutical oligonucleotide products in various stages of clinical development for the treatment of an assortment of life-threatening diseases.¹⁻³ For in vivo applications, all compounds in phase III trials are modified from the natural structure (1; FIG. 1) to make them more persistent within the body.

These late-phase trials have utilised, or are utilising, thiophosphate (or phosphorothioate) modification of oligonucleotides in which an oxygen is substituted by a sulfur atom (2) so as to make the resulting analogue more resistant to cleavage by enzymes.

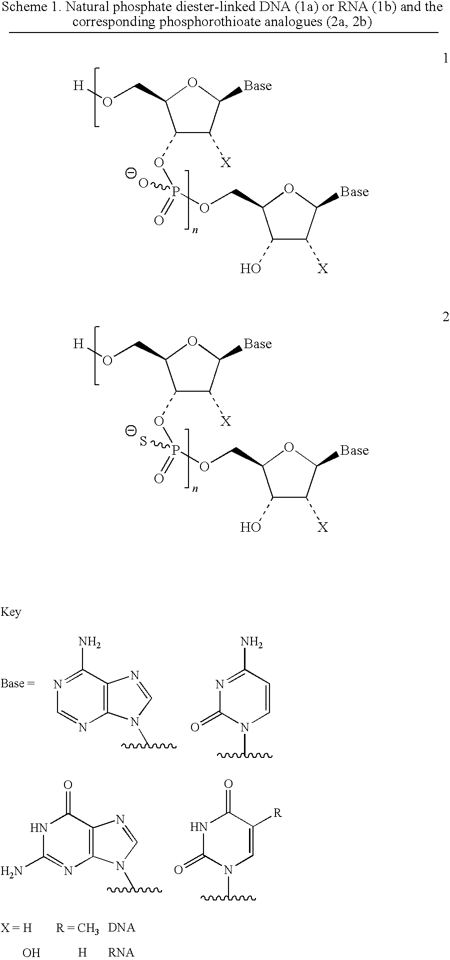

The first internucleotide thiophosphate was prepared by Eckstein.⁴ Workers in the Stec group first developed methods for the preparation of thiophosphate-modified oligonucleotides using solid-phase chemistry which incorporated a sulfurisation step.^{5,6} Sulfurisation of either a phosphite triester on solid-support or an H-phosphonate-linked oligonucleotide was originally performed using elemental sulfur (Scheme 2).

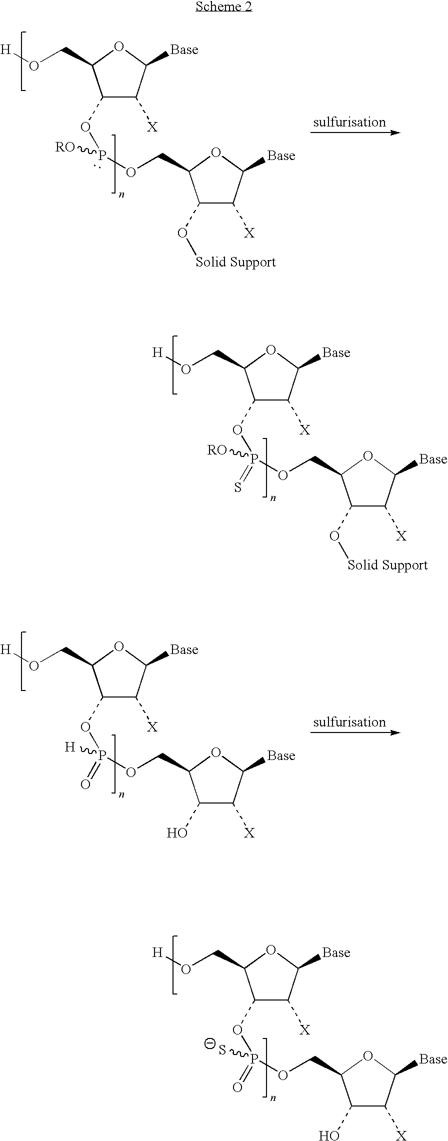

Thus, originally, elemental sulfur was used for this step but its lack of solubility in all solvents at room temperature except in carbon disulfide and incomplete sulfurisation using such conditions has led to the development of several different proprietary sulfurising agents which are used instead of sulfur (e.g. Scheme 3).⁷⁻¹⁰ All of these agents require complex synthesis, many are not stable towards degradation, although typical sulfurisation efficiencies are 99-99.5%.

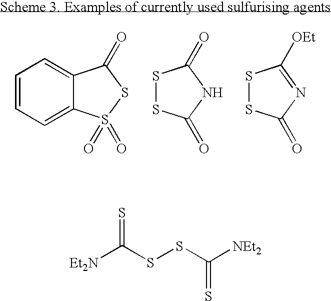

Since their initial description, thiophosphate analogues of other nucleic acids including oligonucleotide phosphate monoesters (3; Scheme 4), nucleoside cyclic phosphate diesters (4) and nucleoside triphosphates (5) have all been prepared and utilised in a wide variety of applications including: terminal labelling of oligonucleotides;^{11,12} time-resolved crystallography;¹³ brain imaging;¹⁴ and massively parallel analysis of the structural requirements for functional nucleic acids.¹⁵

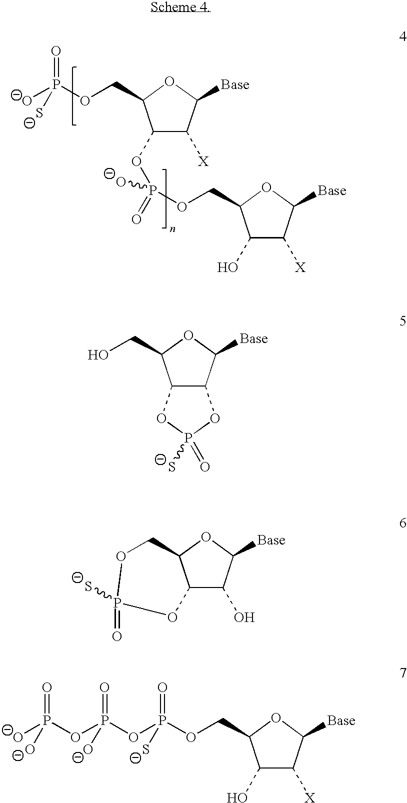

Phosphorothioate modified nucleic acids Selenophosphates (Scheme 5; Y═Se) have also been prepared—most recently using triphenylphosphine selenide¹⁶ or via an organometallic route.¹⁷ Such reagents are of particular interest in the determination of nucleic acid structures using X-ray crystallography.¹⁸⁻²² Also known is non-nucleosidic tellurophosphate diester preparation.²³

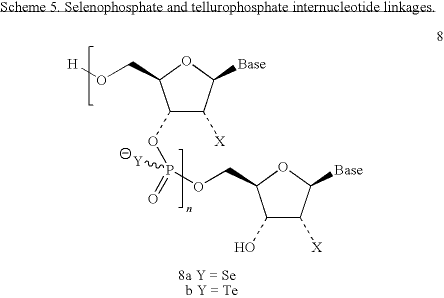

Selenium has also been found to be an essential nutrient for mammals. The metabolic role of selenium is underlined by reactions catalyzed by selenium-dependent enzymes, in which selenium is linked to cysteine. Several organoselenium compounds have been studied from the point of view of anticancer, antihistamine, or anti-inflammatory properties²⁹.

However, the use of elemental selenium³⁰ or potassium selenocyanate^{31,32} as selenium sources has generated experimental problems due to the low solubility and reactivity of these compounds. A new selenium-transfer reagent, 3H-1,2-benzothiaselenol-3-one has been investigated.^{33,34}

We are unaware of any disclosures in the literature of the use of ionic liquids as reaction media utilizing Group 16 elements as reactants, although we are aware of a disclosure of the use of ionic liquids for oligonucleotide synthesis (U.S. Pat. No. 6,852,850 B2). We are also aware of reports describing the application of ionic liquids to the desulfurisation of fuel or flue-gas^{24,25}, particularly to remove sulfur-containing compounds such as sulfur dioxide or thiophene. Other reports have been made of the manipulation of nucleosides in ionic liquids (but not of nucleotides).²⁶⁻²⁸

The present invention is based upon the finding that reactions involving the introduction of atoms of Group 16 elements may be effectively carried out using a reaction medium comprising an ionic liquid, and presenting the Group 16 element in free (i.e. elemental) form as reagent. By “elemental form”, is meant that the Group 16 element (sulfur, selenium or tellurium) is not combined in a molecule containing any other element. Thus, in the case of sulfur, the sulfur may be in the S₈ elemental form, i.e. in the form of octomeric molecules S₈.

According to one aspect of the invention, there is provided a process for carrying out a reaction between a Group 16 element other than oxygen and an organic or inorganic compound, said Group 16 element being in elemental form, characterised in that the reaction medium in which the reaction takes place comprises at least one ionic liquid.

Preferably in accordance with the invention, the reaction medium comprises, in admixture, (a) an organic ionic liquid and (b) sulfur, selenium or tellurium.

It has been found that with certain ionic liquids, at least a portion of the group 16 element (particularly sulfur) may be present in solution in the ionic liquid. The proportion may be as high as 0.05 g g⁻¹ when measured at 110° C., particularly as high as 0.10 g g⁻¹. With certain ionic liquids, concentrations as high as, particularly 0.40 g g⁻¹ and even as high as 0.70 g g⁻¹ have been observed.

Use of ionic liquids which comprise at least one species of cation, and at least one species of soft anion has been found to be particularly advantageous, in view of their ability to dissolve Group 16 elements, especially sulfur (and also selenium).

The principle of hard and soft ions is well-known in chemistry (see Advanced Organic Chemistry, March J; and d-Block Chemistry, Winter M. J. Soft ions are those with low electronegativity and high polarizability. In contrast, hard ions have high electronegativity and low polarizability, for example, [SO₃OR]⁻.

Preferably, the soft anion is aromatic, basic or both aromatic and basic.

In preferred embodiments, the soft anion may be selected from: [S₂CNR₂]⁻, [S₂CSR]⁻, and [S₂COR]⁻, wherein R may be hydrogen, a C₁ to C₄₀ straight chain or branched alkyl group, a C₃ to C₈ cycloalkyl group, or a C₅ to C₁₀ aryl group, and wherein said alkyl, cycloalkyl or aryl groups may be unsubstituted, or substituted by one to three groups selected from: C₁ to C₆ alkoxy, C₆ to C₁₀ aryl, CN, OH, SH, NO₂, C₇ to C₃₀ aralkyl or C₇ to C₃₀ alkaryl.

More preferably, R is selected from a C₁ to C₁₀ straight chain or branched alkyl group, a C₅ to C₇ cycloalkyl group, or a C₅ to C₈ aryl group, wherein said alkyl, cycloalkyl or aryl groups are unsubstituted or may be substituted by one to three groups selected from: C₁ to C₆ alkoxy, C₆ to C₈ aryl, CN, OH, SH, NO₂, C₈ to C₁₅ aralkyl or C₈ to C₁₅ alkaryl.

Still more preferably, R is selected from a C₁ to C₆ straight chain or branched alkyl group a C₅ to C₆ cycloalkyl group, or a C₅ to C₆ aryl group, wherein said alkyl, cycloalkyl or aryl groups are unsubstituted or may be substituted by one to three groups selected from: C₁ to C₆ alkoxy, C₆ to C₈ aryl, CN, OH, SH, NO₂, C₈ to C₁₅ aralkyl or C₈ to C₁₅ alkaryl.

Further examples of R include:

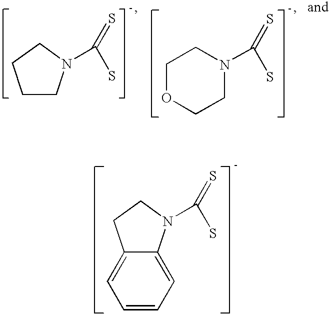

The soft anion may also be selected from: [O₂CR]⁻ wherein R is hydrogen, a C₁ to C₄₀ straight chain or branched alkyl group, and may be substituted by one to three OH groups. Preferably R is a C₁ to C₆ straight chain alkyl group substituted by one OH group. More preferably, R is —CH(OH)CH₃. Alternatively, R may be C₁ to C₁₅ unsubstituted straight chain alkyl, preferably C₅ to C₁₂ alkyl and most preferably C₉ alkyl.

The soft anion may also be selected from: [SO₃R]⁻ wherein R is a C₁ to C₄₀ straight chain or branched alkyl group substituted by one to three SH groups. Preferably, R is a C₁ to C₆ straight chain alkyl group substituted by one OH group. More preferably, R is —CH₂—CH₂—SH.

Other examples include: [S₂CSBu]⁻, [(S₂CSCH₂CH₂)S]²⁻, [(S₂CSCH₂)]²⁻, [S₂CNEt₂]⁻, [S₂CN(CHMe₂)₂]⁻, [S₂CN(CH₂)₂O(CH₂)₂]⁻, [S₂CN(CH₂)₄]⁻, [S₂COMe]⁻, [S₂COEt]⁻, [S₂COCHMe₂]⁻, [S₂COBu]⁻, [SO₃(CH₂)₂SH]⁻ and [S₂COPent]⁻ in which elemental sulfur has an unexpectedly high solubility.

A further example is [N(CN)₂]⁻.

The cation of ionic liquids used in accordance with this invention may comprise or consist of a heterocyclic ring structure selected from imidazolium, pyridinium, pyrazolium, thiazolium, isothiazolinium, azathiazolium, oxothiazolium, oxazinium, oxazolium, oxaborolium, dithiazolium, triazolium, selenozolium, oxaphosholium, pyrollium, borolium, furanium, thiophenium, phospholium, pentazolium, indolium, indolinium, oxazolium, isooxazolium, isotriazolium, tetrazolium, benzofuranium, dibenzofuranium, benzothiophenium, dibenzothiophenium, thiadiazolium, pyrimidinium, pyrazinium, pyridazinium, piperazinium, piperidinium, morpholinium, pyranium, annolinium, phthalazinium, quinazolinium, quinazalinium, quinolinium, isoquinolinium, thazinium, oxazinium, azaannulenium and pyrrolidinium.

More preferably, the cation comprises or consists of a heterocyclic ring structure selected from imidazolium, pyridinium, pyrazolium, thiazolium, pyrimidinium, piperazinium, piperidinium, morpholinium, quinolinium, isoquinolinium and pyrrolidinium.

The invention also provides, according to a further aspect thereof, a reaction medium comprising an organic ionic liquid and at least one Group 16 element other than oxygen, said Group 16 element being in elemental form. Preferably the reaction medium comprises in admixture (a) an organic ionic liquid and (b) sulfur, selenium or tellurium, especially sulfur. Most preferably at least a portion of the Group 16 element is in solution in the ionic liquid.

The term “ionic liquid” refers to a liquid that is capable of being produced by melting a solid, and when so produced, consists solely of ions. Ionic liquids may be derived from organic salts, especially salts of heterocyclic nitrogen-containing compounds, and such ionic liquids are particularly preferred for use in the processes of the present invention. Ionic liquids may be regarded as consisting of two components, which are a positively charged cation and a negatively charged anion.

An ionic liquid may be formed from a homogeneous substance comprising one species of cation and one species of anion, or can be composed of more than one species of cation and/or anion. Thus, an ionic liquid may be composed of more than one species of cation and one species of anion. An ionic liquid may further be composed of one species of cation, and one or more species of anion.

Thus, in summary, the term “ionic liquid” as used herein may refer to a homogeneous composition consisting of a single salt (one cationic species and one anionic species) or it may refer to a heterogeneous composition containing more than one species of cation and/or more than one species of anion. For the purposes of the present invention, it is preferred that the anion species of the ionic liquid comprises a halide, i.e. F⁻, Cl⁻, Br⁻ or I⁻. Preferably, the ionic liquid employed in the present invention is composed of a single species of halide anions, with Cl⁻ being particularly preferred.

The term “ionic liquid” includes compounds having both high melting temperature and compounds having low melting points, e.g. at or below room temperature (i.e. 15-30° C.). The latter are often referred to as “room temperature ionic liquids” and are often derived from organic salts having pyridinium and imidazolium-based cations.

A feature of ionic liquids is that they have particularly low (essentially zero) vapour pressures. Many organic ionic liquids have low melting points (e.g. less than 100° C., particularly less than 100° C., and around room temperature, e.g. 15-30° C. and some have melting points well below 0° C. For the purposes of the present invention, it is desirable that the organic ionic liquid has a melting point of 250° C. or less, preferably 150° C. or less, more preferably 100° C. or less and even more preferably 80° C. or less, although any compound that meets the criteria of being a salt (consisting of an anion and cation) and which is liquid at or near the reaction temperature, or exists in a fluid state during any stage of the reaction can be defined as an organic ionic liquid especially suitable for use in the process of the present invention.

Ionic liquids useful for preparing the reaction medium of the present invention include those comprising an imidazolium, pyridinium, pyridazinium, pyrazinium, oxazolium, triazolium, pyrazolium, pyrrolidinium, piperidinium, tetraalkylammonium or tetraalkylphosphonium salt.

Ionic liquids for use in the present invention include salts (preferably halide salts, and especially chloride salts) of imidazoles, pyridines, pyridazines, pyrazines, oxazoles, triazoles or pyrazoles. Preferred ionic liquids for use in the present invention are imidazolium, pyridinium, pyridazinium, pyrazinium, oxazolium, triazolium or pyrazolium halide salts.

Especially preferred ionic liquids are halide salts of an alkylated or polyalkylated compound of pyridine, pyridazine, pyrimidine, pyrazine, imidazole, pyrazole, oxazole or triazole.

Also preferred ionic liquids for use in the present invention are those comprising an imidazolium, pyridinium, pyridazinium, pyrazinium, oxazolium, triazolium, pyrazolium, pyrrolidinium or piperidinium halide salt, with imidazolium halide salts (particularly chloride) being particularly preferred.

Thus, ionic liquids suitable for use in the present invention include those selected from a compound of formula:

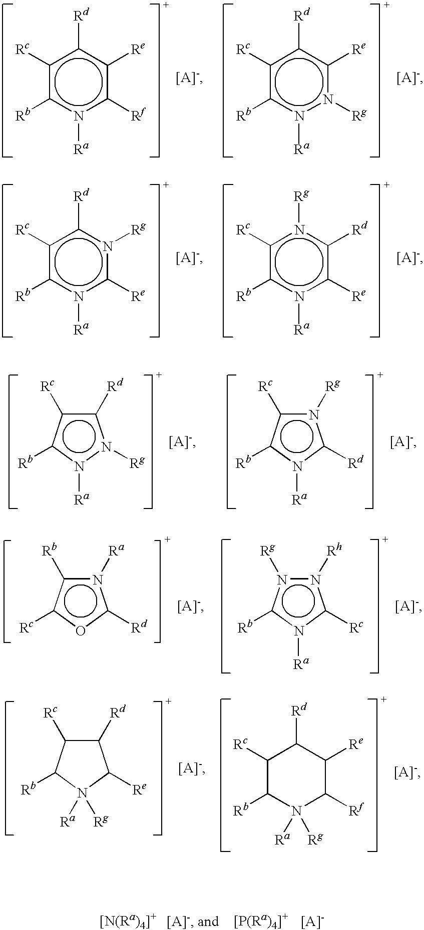

wherein
each R^{a} is independently selected from a C₁ to C₄₀ linear or branched alkyl or a C₃ to C₈ cycloalkyl group, wherein said alkyl or cycloalkyl group which may be substituted by one to three groups selected from: C₁ to C₆ alkoxy, C₆ to C₁₀ aryl, C₁ to C₃₀ aralkyl and C₁ to C₃₀ alkaryl;
each R^{b}, R^{c}, R^{d}, R^{e}, R^{f}, R^{g} and R^{h} can be the same or different and are each independently selected from H or any of the R^{a} groups as defined above; and
[A]⁻ represents a monovalent anion (halide is preferred, and chloride is especially preferred).

Also preferred are ionic liquids selected from a compound of formula:

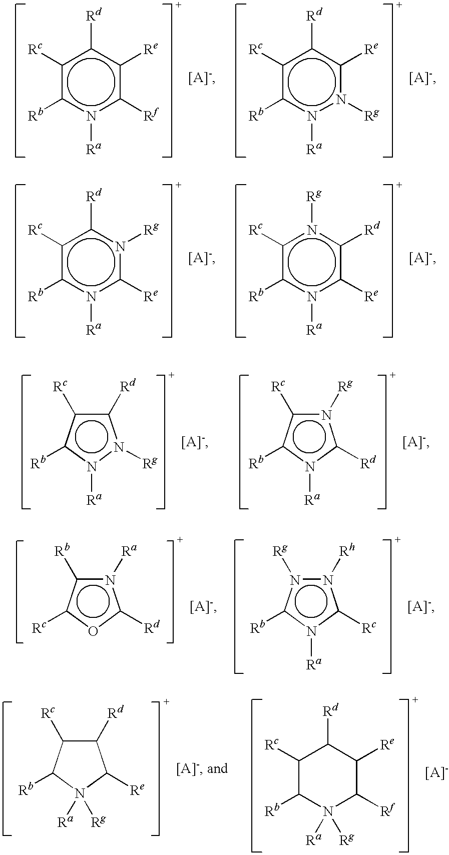

wherein R^{a}-R^{h} and [A]⁻ are as defined above.

Also preferred are ionic liquids selected from a compound of formula:

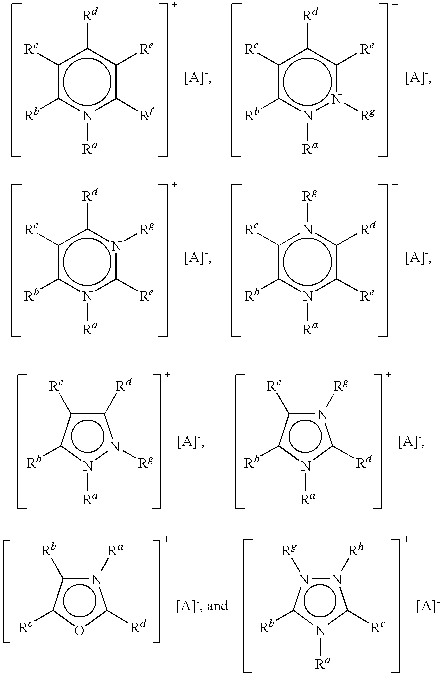

wherein R^{a}-R^{h} and [A]⁻ are as defined above.

Also preferred are ionic liquids selected from a compound of formula:

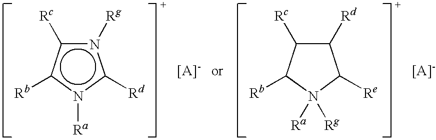

wherein [A]⁻, R^{a}, R^{b}, R^{c}, R^{d}, R^{e} and R^{g} are as defined above.

Also preferred are protonated ionic liquids, for example, alkylimidazoliums.

Imidazole-based ionic liquids selected from a compound of formula:

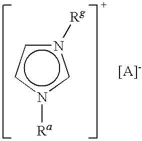

wherein R^{a}, R^{g} and [A]⁻ are as defined above are especially useful.

In the above formulae, it is preferred that each R^{a} represents C₁ to C₄₀ (preferably C₁ to C₂₀, even more preferably C₁ to C₈) linear or branched alkyl.

Also; in the above formulae, it is preferred that each R^{g} and R^{h} represents C₁ to C₄₀ (preferably C₁ to C₂₀, more preferably C₁ to C₈) linear or branched alkyl. Particularly preferred are ionic liquids of the above formulae wherein R^{b}, R^{c}, R^{d}, R^{e}, R^{f}, R^{g} and R^{h} each represents hydrogen.

A further preferred group of ionic liquids are those having the above formulae wherein R^{a}, R^{g} and R^{h} each represents a C₁-C₂₀ alkyl group.

The anion [A]⁻ in the ionic liquids of the above formulae is preferably halide, i.e. F⁻, Cl⁻, Br⁻ or I⁻. Cl⁻ or Br⁻ are preferred halide anions, and Cl⁻ is especially preferred.

Specific examples of suitable ionic liquids for the reaction mediums of the present invention include:
methylimidazolium halide
1-butyl-3-methylimidazolium halide
1-octyl-3-methylimidazolium halide
1-decyl-3-methylimidazolium halide
1-dodecyl-3-methylimidazolium halide
1-ethyl-3-methylimidazolium halide
1-hexyl-3-methylimidazolium halide
1-hexylpyridinium halide
1-octylpyridinium halide

In the above examples of suitable ionic liquids, it is preferred that the halide anion is chloride.

Good results have been obtained when 1-ethyl-3-methylimidazolium chloride or 1-butyl-3-methylimidazolium chloride are employed as starting materials for the reaction mediums of the present invention.

In a further preferred embodiment, Cat⁺ is [C_{6,6,6,14}P]⁺.

More preferably where Cat⁺ is [C_{6,6,6,14}P]⁺, the anion X⁻ may be selected from:

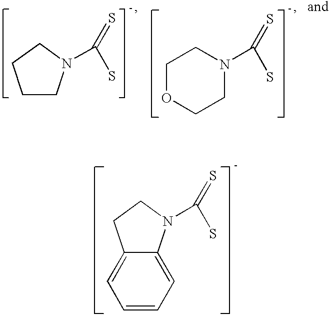

Also X⁻ may be [C₁₀O₂]⁻ or [N(CN)₂]⁻.

### Preparation of Internucleotide Phosphorothioate Linkages

The method of the invention is of particular applicability to the production of phosphorothioate-modified oligonucleotides. In this regard, compounds which may be used as reactants/starting materials in method of the invention include:

(1) H-phosphonates represented by the general formula:

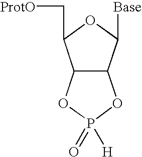

wherein “Prot” represents a hydroxy-protecting group and “Base” represents a nucleotide base, e.g. a naturally occurring base such as adenine, thymine, cytosine, guanine or uracil, or a base analogue, or a protected base, or a protected base analogue;
(2) Phosphite esters represented by the general formula:

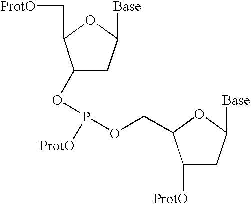

wherein each of the entities “Base” and “Prot” (which may be the same or different) are as defined above,
(3) Supported compounds of the above, wherein the support may be either solid phase, e.g. silica, polystyrene; or liquid-phase, e.g. polyethylene glycol.

The present invention will now be described in more detail in the following examples

### I. EXAMPLES 1 TO 3

In these examples, sulfur, selenium and tellurium were all reacted with triphenylphosphine in order to asses the effect of using ionic liquids as, or as a component of, the reaction medium. It was found that the reactions proceeded smoothly to give the corresponding triphenylphosphine sulfide, triphenylphosphine selenide and triphenylphosphine telluride in good yield. The reactivity of the 3 elements was in the order S>Se>Te. Even though the solubility of these elements was fairly low (<5 mol %), this level of solubility was sufficient for the reaction to proceed.

### EXAMPLE 1

Sulfur (0.20 g, 6.2 mmol) was added to a solution of triphenylphosphine (0.81 g, 3.1 mmol) in triisobutylmethylphosphonium 4-methylbenzenesulfonate [Buⁱ₃PCH₃][OTs] at room temperature. This mixture was heated gently (to 80° C.) with stirring for 10 minutes. On cooling a clear yellow solution was obtained with a precipitate of excess sulfur. The solution was decanted from the sulfur and diluted with ethyl acetate:Hexane (1:4 mixture) and placed in the freezer (−20° C.). A white solid precipitated from the solution and was filtered off. The solid was found to be 0.77 g of triphenylphosphine sulfide by NMR (77% isolated yield).

### EXAMPLE 2

Selenium (0.158 g, 2.0 mmol) was added to a solution of triphenylphosphine (0.524 g, 2.0 mmol) in 1-ethyl-3-methylimidazolium ethylsulfate [C₂ mim][EtSO₄] (2.0 g) at room temperature. This mixture was heated gently (to 150° C.) with stirring for 18 hours. The yield was determined by ³¹P, ¹³C and ¹H NMR spectra of the ionic liquids solution after 4 and 18 hours. Gave 96% yield after 4 hours and no observed decomposition of the ionic liquid.

### EXAMPLE 3

Tellurium (0.255 g, 2.0 mmol) was added to a solution of triphenylphosphine (0.524 g, 2.0 mmol) in 1-ethyl-3-methylimidazolium ethylsulfate [C₂ mim][EtSO₄] (2.0 g) at room temperature. This mixture was heated gently (to 150° C.) with stirring for 18 hours. The yield was determined by ³¹P, ¹³C and ¹H NMR spectra of the ionic liquids solution after 4 and 18 hours. Gave 40% yield after 4 hours and no observed decomposition of the ionic liquid and 70% yield after 18 hours.

### II. EXAMPLE 4

### Further Reactions in Solution

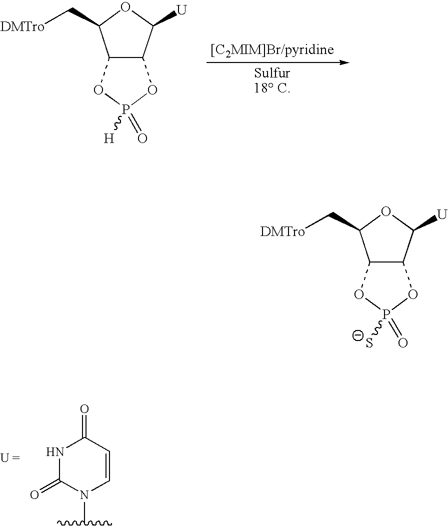

### EXAMPLES 5 AND 6

### Reactions on Solid-Support

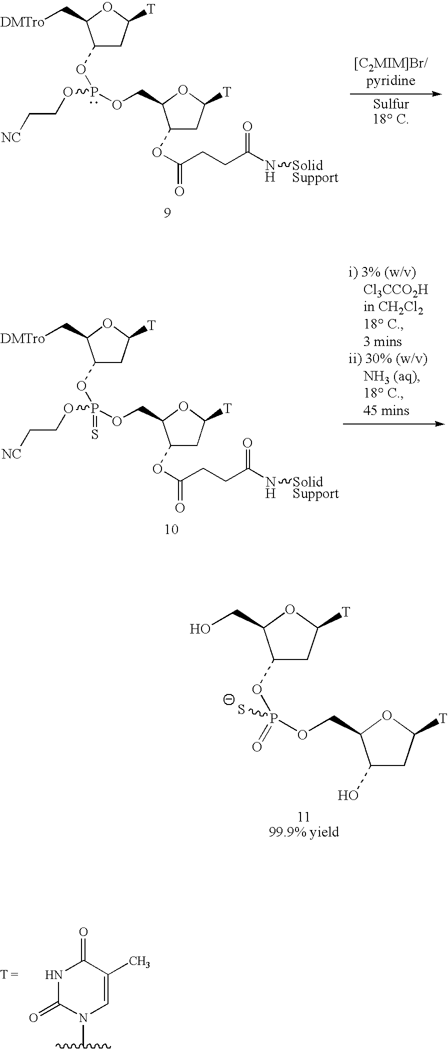

Sulfur was reacted with a solid-supported internucleoside phosphite triester (9) under ambient conditions to investigate the use of an ionic liquid as a reaction medium. It was found that the reactions proceeded smoothly to give the corresponding thiophosphate triester in yields up to 99.9% in a solvent-dependent fashion.

### EXAMPLE 5

A suspension of sulfur (100 mg, 3 mmol) in ethylmethylimidazolium bromide (3.5 g) and anhydrous pyridine (1.5 g) was heated at 50° C. under argon for 2 minutes. The suspension was allowed to cool to room temperature and delivered to 9 (200 nmol) through a 0.45 Hm PTFE syringe filter over 5 minutes under ambient conditions. The supported product (10) was then washed with argon (0.2 mL), anhydrous pyridine (5 mL), anhydrous acetonitrile (5 mL)' and argon (0.5 mL). The support was then deprotected using 3% (w/v) trichloroacetic acid followed by concentrated aqueous ammonia at room temperature for 45 minutes.

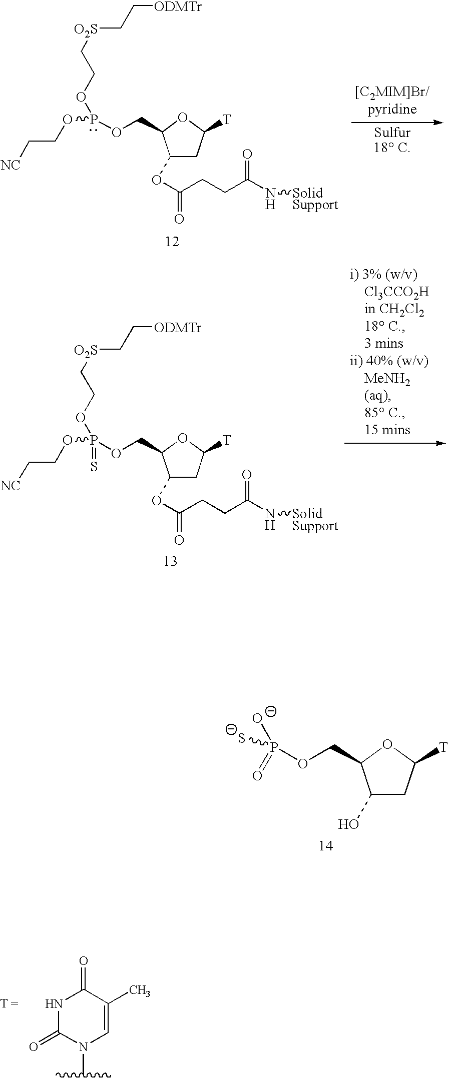

### EXAMPLE 6

A suspension of sulfur (100 mg, 3 mmol) in ethylmethylimidazolium bromide (3.5 g) and anhydrous pyridine (1.5 g) was heated at 50° C. under argon for 2 minutes. The suspension was allowed to cool to room temperature and delivered to 12 (200 nmol) through a 0.45 μm PTFE syringe filter over 5 minutes under ambient conditions. The supported product (13) was then washed with argon (0.2 mL), anhydrous pyridine (5 mL), anhydrous acetonitrile (5 mL) and argon (0.5 mL). The support was then deprotected using 3% (w/v) trichloroacetic acid followed by 40% (w/v) aqueous methylamine at 85° C. for 15 minutes.

### III. EXAMPLES 7 TO 20

### Ionic Liquids v. Beaucage Reagent

### Solvents and Reagents

Acetonitrile (diluent grade) was purchased from Transgenomic; 3H-1,2-benzodithiol-3-one-1,1-dioxide (Beaucage reagent) was purchased from Aldrich; pyridine (99.7%, Aldrich) was refluxed and distilled from calcium hydride and used within 24 hours; triethylamine (99%; Sigma) and acetic acid (ACS grade; Aldrich) were both used without further purification; 18 MΩ water was obtained using an Elga Maxima water purification system; ionic liquids were used as supplied without purification.

### Reverse Phase High Performance Liquid Chromatography (RP-HPLC)

HPLC analysis was performed using an Agilent 1100 equipped with a Zorbax Eclipse XDB-C₁₈ column (4.6×150 mm, 5 μm) eluting with the following gradient of acetonitrile in 100 mM triethylammonium acetate (pH 6.5) in 95:5 H₂O:MeCN:: 0%, 0-5 mins; 0-20% 5-35 mins; 20-100%, 35-45 mins 100% 45-50 mins; 100-0% 50-60 mins. The peaks were monitored at 270 nm.

### Preparation of Tp(s)T

Dimer syntheses were carried out on a Beckman 100M DNA synthesizer utilizing a 1000 nmol standard cycle with 200 nmol dT-columns (1000 A, CPG). The automated cycle was paused immediately following the coupling step before delivery of any further reagents. Individual columns were removed from the synthesizer, washed with anhydrous acetonitrile (5 mL), purged with argon (0.5 mL), treated with sulfurisation reagent (0.7 mL; see below) for 1, 2, 4, 5 or 8 minutes (but principally 5 mins), purged with argon (0.2 mL), washed with dry pyridine (5 mL), purged with argon (0.5 mL), washed again with acetonitrile (5 mL) and purged with argon (0.5 mL). The columns were replaced on the synthesizer for the final detritylation step without performing any intermediate capping or oxidation steps.

The product was released from the solid support by treatment with concentrated aqueous ammonia (0.7 mL) over 45 minutes using the “double-syringe” technique.^{[18]} The ammonia was removed by concentration in vacuo and the sample solutions diluted to 1 mL with 100 mM triethylammonium acetate (pH 6.5 (in 95:5 H₂O:MeCN prior to analysis by RP-HPLC).

Sulfurising reagents were prepared by heating a mixture of sulfur (100 mg, 3 mmol) and the ionic liquid (3.5 g) at 80° C. under argon for 3 minutes. The suspension were then allowed to cool to room temperature and dry pyridine (1.5 mL) was added. The resultant suspension was further heated at 50° C. for 2 minutes and allowed to cool to room temperature. The suspensions were filtered through 0.45 μm nm PTFE syringe filters (Whatman) prior to delivery to the column.

The ionic liquids utilized in this studies were: [emim]Br (A); [emim][EtSO₄] (B); [emim][MeCH(OH)CO₂] (C); [bmim][BF₄] (D); [N_{2.2.2.2}][NTf₂] (E); [N_{2.2.2.1}][MeSO₄] (F); [N(Me)Pr₃][OTs] (G); [P_{4.4.4.4}][NO₃] (H); [P_{6.6.6.14}][N(CN)₂] (I); [P_{6.6.6.14}][NonCO₂] (J);

The same procedure may also be used in the preparation of Tp(Se)T and Tp(Te)T.

### Results

HPLC analysis of standards of TpT (prepared using a standard synthesis cycle with 50 mM iodine in 8:1:1 THF:pyridine:water as oxidant), Tp(s)T (prepared using Beaucage reagent) and the product of iodine-mediated desulfurisation of Tp(s)T prepared using sulfurisation mixture I, enabled the following peak retention times to be assigned (Table 1)

TABLE 1
Peak retention times identified from HPLC analysis

| Dinucleoside | | |
|---|---|---|
| thiophosphate | TpT | Tp(s)T |
| Retention | 13.783 | 17.407 |
| time(s) (min) | | 18.611 |

The current industry standard for such sulfurisations is Beaucage reagent (92 mM in anhydrous MeCN) and compassion with this standard was made.

| | | | % Tp(s)T |
|---|---|---|---|
| | Reagent for | Time of reaction | compared with |
| Example | sulfurisation | (mins) | total Tp(s)T + TpT |
| | Beaucage (standard) | 5 | 98.2600 |
| 7 | A | 1 | 99.3544 |
| 8 | A | 2 | 99.8885 |
| 9 | A | 4 | 99.8846 |
| | 10 | 5 | 99.9149 |
| 11 | A | 8 | 99.7246 |
| 12 | B | 5 | 94.0843 |
| 13 | C | 5 | 89.1779 |
| 14 | D | 5 | 93.3706 |
| 15 | E | 5 | 98.7916 |
| 16 | F | 5 | 98.7486 |
| 17 | G | 5 | 50.8422 |
| 18 | H | 5 | 4.8669 |
| 19 | I | 5 | 97.6191 |
| 20 | J | 5 | 95.0314 |

### IV. EXAMPLES 21 TO 34

### Preparation of Tp(s)T

### Preparation of Tp(s)T

Selenophosphate synthesis was carried out (as above) with a solid-support using a DNA synthesizer (Beckman 1000M). The synthesis utilized standard 200 nmol dT-CE columns. A 1000 nmol scale standard cycle was used as for Tp(Sc)T above. Selenium-transfer was done manually using a selenium-containing mixture composition: 0.1 g selenium suspended in 2 ml dry ionic liquid. The suspension was heated to and maintained at 90° C. under nitrogen for 6 hours. To this was added 1 ml dry pyridine (freshly distilled and heated for 15 minutes at 90° C. under nitrogen and then filtered through a 45 μm PTFE filter).

The automated cycle was paused following the coupling step, the column was removed from the synthesizer, washed with 5 ml anhydrous acetonitrile, purged with 0.5 ml argon, reacted with 0.7 ml selenium-transfer reagent (purged with 0.2 ml argon) and prepared as above, over 5 minutes, washed with 5 ml of dry pyridine, purged with 0.5 ml argon, then washed again with 5 ml acetonitrile, purged with 0.5 ml argon and replaced on the synthesizer in order to perform the subsequent detritylation step.

The selenophosphate product was released from the solid support by treatment with 0.7 ml concentrated ammonia over 45 minutes using the “double syringe” technique. The ammonia was removed by concentration in vacuo and the samples made up to 1 ml with buffer comprising 100 mM triethylammonium acetate in a mixture of acetonitrile/water 5/95 (v/v).

### Results

The samples were analyzed by reversed phase HPLC (Agilent 1100 Series) using a Zorbax Eclipse XDB-C₁₈ column (4.6×150 mm, 5 μm) eluting with a gradient (shown below) of buffer in acetonitrile.

| Time (min) | Buffer (%) | Acetonitrile (%) |
|---|---|---|
| 0 | 100 | 0 |
| 5 | 100 | 0 |
| 35 | 80 | 20 |
| 45 | 0 | 100 |
| 50 | 0 | 100 |
| 60 | 100 | 0 |

Selenium-transfer efficiency was determined as a ratio between selenophosphates and selenophosphates and products of all side reactions involved.

| | | Tp(Se)T/Σ | Tp(Se)T/Σ |
|---|---|---|---|
| Example | Ionic liquid | 1^{st} run | 2^{nd} run |
| 21 | [P₆₆₆₍₁₄₎]⁺ [C₁₀O₂]⁻ | 87.6110 | 88.7962 |
| 22 | [P₆₆₆₍₁₄₎]⁺ [C₁₂O₂]⁻ | 7.1712 | 89.7774 |
| 23 | [P₆₆₆₍₁₄₎]⁺ [N(CN)₂]⁻ | 93.1257 | |
| 24 | 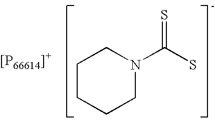 | 88.2647 | 89.9252 |
| 25 | 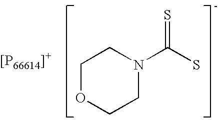 | 85.9838 | 83.1798 |
| 26 | 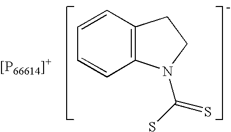 | 85.2898 | |

The stability of selenophosphates synthesized with selenium-ionic liquids/pyridine mixtures was determined as follows: the deprotected samples of selenophosphates were analyzed under similar conditions after 3 weeks. A small amount of selenium was “washed-out”.

| | | Tp(Se)T/Σ | Tp(Se)T/Σ |
|---|---|---|---|
| Example | Ionic liquid | initial | after 3 weeks |
| 27 | [P₆₆₆₍₁₄₎]⁺ [C₁₀O₂]⁻ | 87.6110 | 86.2848 |
| 28 | [P₆₆₆₍₁₄₎]⁺ [N(CN)₂]⁻ | 93.1257 | 93.0537 |
| 29 | 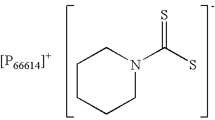 | 88.2647 | 88.8118 |
| 30 | 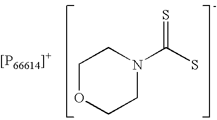 | 85.9838 | 85.8837 |

Also investigated was the stability of selenium-transfer mixtures consisting of selenium dissolved in ionic liquids/pyridine 2/1 (v/v). The selenium-ionic liquid/pyridine mixtures utilised for selenophosphate synthesis was kept for 3 weeks in dark conditions, at room temperature. The selenium-transfer mixtures appear to degrade over time (see [P₆₆₆₍₁₄₎]⁺ [C₁₀O₂]⁻ and [P₆₆₆₍₁₄₎]⁺ [N(CN)₂]⁻).

| | | | Tp(Se)T/Σ |
|---|---|---|---|
| | | | after 3 weeks |
| | | | using the |
| | | | same selenium- |
| Exam- | | Tp(Se)T/Σ | transfer |
| ple | Ionic liquid | initial | mixture |
| 31 | [P₆₆₆₍₁₄₎]⁺ [C₁₀O₂]⁻ | 87.6110 | 27.0320 |
| 32 | [P₆₆₆₍₁₄₎]⁺ [N(CN)₂]⁻ | 93.1257 | 3.1997 |
| 33 | 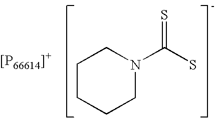 | 88.2647 | 89.4096 |
| 34 | 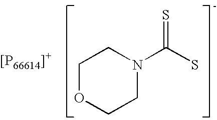 | 85.9838 | 90.4344 |

### V EXAMPLES 35 TO 126

### Further Phosphorothioates and Selenophosphates

Both oligonucleotide phosphorothioates and selenophosphates were prepared using the methods described above.

In 1980 Ogilvie used elemental selenium in order to prepare dinucleotide selenophosphate. The method is relatively complicated because involves centrifugation, filtration, removal of solvent in which the starting material (protected dinucleotide) was dissolved. The general preparation of oligonucleotide selenophosphates in solid phase uses KSeCN and the selenium-transfer step is performed out of machine, using a manual procedure. Using selenium dissolved in ionic liquids it is possible to prepare selenophosphates using solid phase synthesis and, the efficiency of selenium-transfer is much higher.

### Preparation

In the present examples, the dinucleotide phosphorothioates, and selenophosphates containing mixed sequences were released from the solid support by treatment with 0.5 ml CH₃NH₂ over 30 minutes at 65° C., and then the controlled-pore glass support washed with an additional 0.3 ml water. The amine was removed by concentration in vacuo and the samples made up to 1 ml with a buffer comprising in 100 mM triethylammonium acetate with a mixture of acetonitrile/water 5/95 (v/v) for Tp(S)T, and Tp(Se)T, deprotection was done with 0.7 ml concentrated ammonia over 45 minutes at room temperature using “double syringes” technique, then ammonia was removed under vacuum as disclosed in the experiments above.

The samples were then analyzed by reversed phase HPLC.

### Results

Chalcogen-transfer efficiency was determined as the ratio between derivatized dinucleotide and products of all side reactions involved. As [EMIM]⁺Br⁻ (has been demonstrated to produce good yields for phosphorothioate, S₈ in [EMIM]⁺Br⁻ was employed as the sulfur-transfer mixture for the preparation of mixed sequences.

| | | | Np(S)N/ |
|---|---|---|---|
| | | Np(S)N/ | Σ peaks II |
| Example | Sequence | Σ peaks | RUN |
| 35 | Tp(S)A | 79.2567 | 88.6919 |
| 36 | Tp(S)C | 59.9076 | 89.7041 |
| 37 | Tp(S)G | 69.7300 | 72.5663 |
| 38 | Ap(S)A | 81.6233 | 68.6900 |
| 39 | Ap(S)C | 53.6840 | — |
| 40 | Ap(S)G | 72.9137 | 77.4924 |
| 41 | Ap(S)T | 70.0514 | — |
| 42 | Cp(S)A | 63.7692 | 74.4927 |
| 43 | Cp(S)C | 39.1844 | 79.8931 |
| 44 | Cp(S)G | 70.8312 | 70.8276 |
| 45 | Cp(S)T | 64.1034 | 84.1623 |
| 46 | Gp(S)A | 71.3800 | 77.3826 |
| 47 | Gp(S)C | 49.5540 | 70.6498 |
| 48 | Gp(S)G | 73.7238 | — |
| 49 | Gp(S)G | 76.1070 | 82.1872 |

In another set of experiments, [P_{6,6,6,14}]⁺[NonCOO]⁻ was used to synthesise dinucleotide derivatives with mixed sequences.

| | | Np(S)N/(Σ | Np(S)N/(Σ | | |
|---|---|---|---|---|---|
| | | peaks) | peaks) | Np(Se)N/(Σ | NpN/(Σ |
| Example | Sequence | RUN I | RUN II | peaks) | peaks) |
| 50 | Tp(x)A | 89.6400 | 88.6919 | 91.9051 | 90.3365 |
| | | (highest) | | | |
| 51 | Tp(x)C | 82.8970 | 89.7041 | 89.0866 | 85.5633 |
| 52 | Tp(x)G | 69.7892 | 72.5663 | 73.2407 | 86.5916 |
| 53 | Tp(x)T | 86.0761 | — | 88.7962 | 91.1115 |
| 54 | Ap(x)A | 88.0727 | 68.6900 | 91.7143 | 77.6848 |
| 55 | Ap(x)C | 77.6968 | — | 86.3238 | 78.8843 |
| 56 | Ap(x)G | 77.3213 | 77.4924 | 80.2911 | 82.2433 |
| 57 | Ap(x)T | 88.9831 | — | 87.6350 | 91.8434 |
| 58 | Cp(x)A | 80.9338 | 74.4927 | 87.1171 | 92.0366 |
| 59 | Cp(x)C | 65.1416 | 79.8931 | 82.2683 | 90.1333 |
| | | (lowest) | | | |
| 60 | Cp(x)G | 68.8181 | 70.8276 | 76.5393 | 90.0510 |
| 61 | Cp(x)T | 82.1720 | 89.1623 | 86.0729 | 92.2280 |
| 62 | Gp(x)A | 81.6651 | 77.3826 | 81.8844 | 88.5901 |
| 63 | Gp(x)C | 69.7962 | 70.6498 | 84.3774 | 88.9233 |
| 64 | Gp(x)G | 72.9127 | — | 68.9815 | 89.6873 |
| 65 | Gp(x)T | 87.0528 | 82.1872 | 86.6050 | 83.9211 |

The results suggest that phosphorothioates are more easily oxidized than the corresponding selenophosphate as shown by the lower yield. It is also noted that Cp(S)C and Gp(Se)G were synthesized with the lowest yield. A possible explanation is the existence of protecting groups on both the cytidyl- and guanidyl-bases could hinder the access of chalcogen-transfer reagents to the phosphorus centre, or even interact with them. It is also known that removal of protecting groups, especially in the case of modified purine nucleotides, can produce depurination, which would give low yields.

Other ionic liquids were then tested.

| Exam- | | | Np(X)N/ |
|---|---|---|---|
| ple | Ionic Liquid | Sequence | (Σ peaks) |
| 66 67 68 69 | 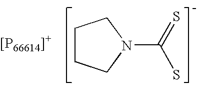 | Tp(Se)T Tp(S)T Cp(S)C Gp(S)G | 70.34 73.57 36.39 |
| 70 71 72 73 | 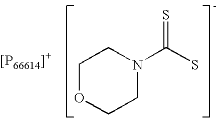 | Tp(Se)T Tp(S)T Cp(S)C Gp(S)G | 88.63 63.42 58.54 7.96 |
| 74 75 76 77 | 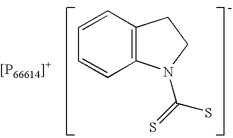 | Tp(Se)T Tp(S)T Cp(S)C Gp(S)G | 81.43 81.92 73.34 |
| 78 | 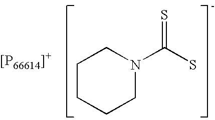 | Tp(Se)T | 87.46 |

For reference, the identification of peaks by HPLC, especially in case of phosphoroselenoate dinucleotides, was done by elemental analysis, co-injection, and mass spectrometry. For mass spectrometry the presence of a specific pattern as shown in FIG. 1, due to the existence of Se isotopes, demonstrates the presence of phosphoroselenoate dinucleotide.

The results below show a comparison between conversion for phosphorothioates newly prepared (the second run), and conversion for old samples previously synthesized:

| | Np(s)N in [P_{666 14}]⁺ | Np(s)N/Σ peaks | Np(s)N/Σ peaks |
|---|---|---|---|
| Example | [C₁₀O₂]⁻/pyridine | 2^{nd} run | 1^{st} run |
| 79 | Ap(s)A | 68.6900 | 88.0727 |
| 80 | Ap(s)C | 84.9071 | 77.6968 |
| 81 | Ap(s)G | 77.4924 | 77.3213 |
| 82 | Ap(s)T | 85.2429 | 88.9831 |
| 83 | Cp(s)A | 74.4927 | 80.9338 |
| 84 | Cp(s)C | 79.8931 | 65.1416 |
| 85 | Cp(s)G | 70.8276 | 68.8181 |
| 86 | Cp(s)T | 84.1623 | 82.1720 |
| 87 | Gp(s)A | 77.3826 | 81.6651 |
| 88 | Gp(s)C | 70.6498 | 69.7967 |
| 89 | Gp(s)G | 72.9374 | 72.9127 |
| 90 | Gp(s)T | 82.1872 | 87.0528 |
| 91 | Tp(s)A | 88.6919 | 89.6400 |
| 92 | Tp(s)C | 89.7041 | 82.8970 |
| 93 | Tp(s)G | 72.5663 | 69.7892 |
| 94 | Tp(s)T | 89.8601 | 86.0761 |

Further tests were carried out to compare conversion for phosphoroselenoates newly synthesized (the second run) and conversion for old samples previously synthesized.

| | Np(Se)N in | | |
|---|---|---|---|
| | [P_{666 14}]⁺ | | |
| | [C₁₀O₂]⁻/ | Np(Se)N/ | Np(Se)N/ |
| Example | pyridine | peaks 2^{nd} run | peaks 1st run |
| 95 | Ap(Se)A | 76.458 | 91.7143 |
| 96 | Ap(Se)C | 83.5952 | 86.3238 |
| 97 | Ap(Se)G | 66.6263 | 80.2911 |
| 98 | Ap(Se)T | 79.1829 | 87.6350 |
| 99 | Cp(Se)A | 80.5913 | 87.1171 |
| 100 | Cp(Se)C | 75.4560 | 82.2683 |
| 101 | Cp(Se)G | 65.0769 | 76.5393 |
| 102 | Cp(Se)T | 82.9348 | 86.0729 |
| 103 | Gp(Se)A | 77.3131 | 81.8844 |
| 104 | Gp(Se)C | 76.8203 | 84.3774 |
| 105 | Gp(Se)G | 52.60815 | 68.9815 |
| 106 | Gp(Se)T | 80.6598 | 86.6050 |
| 107 | Tp(Se)A | 89.2072 | 91.9051 |
| 108 | Tp(Se)C | 91.6885 | 89.0866 |
| 109 | Tp(Se)G | 69.2092 | 73.2407 |
| 110 | Tp(Se)T | 87.4327 | 88.7962 |

The influence of a co-solvent was also investigated, and produced results as shown below:

| | Np(s)N in [P_{666 14}]⁺ | Np(s)N/ |
|---|---|---|
| Example | [C₁₀O₂]⁻/CH₃CN | Σ peaks |
| 111 | Ap(s)A | 88.0358 |
| 112 | Ap(s)C | 84.5405 |
| 113 | Ap(s)G | 75.7420 |
| 114 | Ap(s)T | 88.5310 |
| 115 | Cp(s)A | 80.3349 |
| 116 | Cp(s)C | 87.5404 |
| 117 | Cp(s)G | 73.3329 |
| 118 | Cp(s)T | 87.5459 |
| 119 | Gp(s)A | 86.7976 |
| 120 | Gp(s)C | 86.1920 |
| 121 | Gp(s)G | 76.5708 |
| 122 | Gp(s)T | 83.6270 |
| 123 | Tp(s)A | 92.0256 |
| 124 | Tp(s)C | 93.4411 |
| 125 | Tp(s)G | 82.419 |
| 126 | Tp(s)T | 87.8512 |

From the above it can be seen that:
1. freshness of the deprotecting reagent, CH₃NH₂ or ammonia, is important; and
2. use of CH₃CN as co-solvent gives rise to similar conversions.

### VII. OTHER REACTIONS IN IONIC LIQUIDS

Reactions of chalcogenes (sulfur and selenium) with phosphite triesters (both aliphatic and aromatic) in ionic liquids were studied.

The general reaction of chalcogenes in ionic liquids with phosphite triesters is:

$${( R O )}_{3} P + {S}_{8} {}^{IL} {( R O )}_{3} P = S$$

$$R = Me , Et , {}^{i} Pr , n \text{-} Bu , {Ph \\ ( R O )}_{3} P + Se {}^{IL} {( R O )}_{3} P = Se$$

Because of differences in chemical shifts of phosphorus reagents and reaction products, it is possible to follow the reaction by ³¹P-NMR.

| | (RO)₃P | (RO)₃PS | (RO)₃PSe |
|---|---|---|---|
| R | δ (ppm) | δ (ppm) | δ (ppm) |
| Et | 140 | 68.9 | |
| ⁱPr | 139.9 | 65.7 | |
| n-Bu | 139 | 69.5 | |
| Ph | 126.2 | 54.5 | |

### EXPERIMENTAL

Added to a mixture of sulfur (0.1 g, 3.125 mmol), respectively selenium (0.1 g, 1.266 mmol), and ionic liquid (1.5 mL), the same molar equivalent of phosphite triester was added, as shown below.

| | Amount | Amount |
|---|---|---|
| | required for | required for |
| | preparation of | preparation of |
| (RO)₃P | (RO)₃PS (mL) | (RO)₃PSe (mL) |
| (EtO)₃P | 0.54 | 0.22 |
| (ⁱPrO)₃P | 0.72 | 0.29 |
| (nBuO)₃P | 0.86 | 0.35 |
| (PhO)₃P | 0.82 | 0.33 |

The reaction mixture was kept under nitrogen, at 80° C. for 2 hours, then analyzed by ³¹P NMR using as solvent CDCl₃.

### Results and Discussions

The results are expressed both as conversion and yield. They are calculated from the ^{31P} NMR spectra obtained.

| | | | (RO)₃PS | | (RO)₃PSe | |
|---|---|---|---|---|---|---|
| | | | Conversion | Yield | Conversion | Yield |
| IL | R | | (%) | (%) | (%) | (%) |
| [EMIM]⁺ Br⁻ | R = | Et | | | 80.1 | 90.51 |
| | | ⁱPr | | | 57.26 | 90.05 |
| | | nBu | | | 95.39 | 75.35 |
| | | Ph | | | 100 | 7.84 |
| [BMIM]⁺ | R = | Et | 100 | 87.24 | 94.35 | 85.84 |
| [BF₄]⁻ | | ⁱPr | 100 | 86.60 | 95.92 | 96.12 |
| | | nBu | 100 | 57.21 | 100 | 84.46 |
| | | Ph | 10.30 | 30.49 | 81.23 | 100 |
| [EMIM]⁺ | R = | Et | 100 | 95.67 | 73.30 | 85.49 |
| [EtSO₄]⁻ | | ⁱPr | 91.57 | 100 | 100 | 79.30 |
| | | nBu | 100 | 78.19 | 90.90 | 95.42 |
| | | Ph | 91.55 | 37.49 | 91.23 | 38.92 |
| [BMIM]⁺ | R = | Et | 100 | 94.42 | 100 | 71.53 |
| [HSO₄]⁻ | | ⁱPr | 100 | 91.24 | 100 | 54.12 |
| | | nBu | 100 | 79.36 | 100 | 51.96 |
| | | Ph | 100 | 23.23 | 100 | 73.53 |
| [P_{666 14}]⁺ | R = | Et | 95.02 | 45.04 | 92.91 | 96.39 |
| [C₁₀O₂]⁻ | | ⁱPr | 96.24 | 90.81 | 100 | 76.12 |
| | | nBu | 100 | 52.11 | 100 | 81.38 |
| | | Ph | 95.19 | 71.38 | | |

In general:
1. When [P_{666 14}]⁺ [C₁₀O₂]⁻ is used as solvent, both sulfur and selenium dissolve. Reaction with the phosphite triester takes place in a homogenous system and the reaction product in soluble in the ionic liquid; and
2. A prolonged reaction time at 80° C. may give rise to secondary products of chalcogenophosphate diesters and triesters.

### REFERENCES

1. Crooke, S. T. Antisense strategies. Current Molecular Medicine 4, 465-487 (2004).
2. Scanlon, K. J. Anti-genes: siRNA, ribozymes and antisense. Current Pharmaceutical Biotechnology 5, 415-420 (2004).
3. Sanghvi, Y. S. & Schulte, M. Therapeutic oligonucleotides: The state-of-the-art in purification technologies. Current Opinion in Drug Discovery & Development 7, 765-776 (2004).
4. Eckstein, F. A dinucleoside phosphorothioate. Tetrahedron Letters 13, 1157-1160 (1967).
5. Stec, W. J., Zon, G., Egan, W. & Stec, B. Automated Solid-Phase Synthesis, Separation, and Stereochemistry of Phosphorothioate Analogs of Oligodeoxyribonucleotides. Journal of the American Chemical Society 106, 6077-6079 (1984).
6. Stec, W. J. & Zon, G. Synthesis, Separation, and Stereochemistry of Diastereomeric Oligodeoxyribonucleotides Having a 5′-Terminal Internucleotide Phosphorothioate Linkage. Tetrahedron Letters 25, 5275-5278 (1984).
7. Iyer, R. P., Phillips, L. R., Egan, W., Regan, J. B. & Beaucage, S. L. The Automated Synthesis of Sulfur-Containing Oligodeoxyribonucleotides Using 3H-1,2-Benzodithiol-3-One 1,1-Dioxide As a Sulfur-Transfer Reagent. Journal of Organic Chemistry 55, 4693-4699 (1990).
8. Vu, H. & Hirschbein, B. L. Internucleotide Phosphite Sulfurization With Tetraethylthiuram Disulfide—Phosphorothioate Oligonucleotide Synthesis Via Phosphoramidite Chemistry. Tetrahedron Letters 32, 3005-3008 (1991).
9. Cheruvallath, Z. S., Kumar, R. K., Rentel, C., Cole, D. L. & Ravikumar, V. T. Solid phase synthesis of phosphorothioate oligonucleotides utilizing diethyldithiocarbonate disulfide (DDD) as an efficient sulfur transfer reagent. Nucleosides Nucleotides & Nucleic Acids 22, 461-468 (2003).
10. Xu, Q. H., MusierForsyth, K., Hammer, R. P. & Barany, G. Use of 1,2,4-dithiazolidine-3,5-dione (DtsNH) and 3-ethoxy-1,2,4-dithiazoline-5-one (EDITH) for synthesis of phosphorothioate-containing oligodeoxyribonucleotides. Nucleic Acids Research 24, 1602-1607 (1996).
11. Kuhnast, B., de Bruin, A., Hinnen, F., Tavitian, B. & Dolle, F. Design and synthesis of a new [F-18]fluoropyridine-based haloacetamide reagent for the labeling of oligonucleotides: 2-bromo-N-[3-(2-[F-18]fluoropyridin-3-yloxy)propyl]acetamide. Bioconjugate Chemistry 15, 617-627 (2004).
12. Alefelder, S., Patel, B. K. & Eckstein, F. Incorporation of terminal phosphorothioates into oligonucleotides. Nucleic Acids Research 26, 4983-4988 (1998).
13. Zegers, I. et al. Hydrolysis of a slow cyclic thiophosphate substrate of RNase T1 analyzed by time-resolved crystallography. Nature Structural Biology 5, 280-283 (1998).
14. Hosoi, R. et al. MicroPET detection of enhanced F-18-FDG utilization by PKA inhibitor in awake rat brain. Brain Research 1039, 199-202 (2005).
15. Strobel, S. A. Biochemical identification of A-minor motifs within RNA tertiary structure by interference analysis. Biochemical Society Transactions 30, 1126-1131 (2002).
16. Bollmark, M. & Stawinski, J. A new selenium-transferring reagent-triphenylphosphine selenide. Chemical Communications, 771-772 (2001).
17. Holloway, G. A., Pavot, C., Scaringe, S. A., Lu, Y. & Rauchfuss, T. B. An organometallic route to oligonucleotides containing phosphoroselenoate. Chembiochem 3, 1061-1065 (2002).
18. Buzin, Y., Carrasco, N. & Huang, Z. Synthesis of selenium-derivatized cytidine and oligonucleotides for X-ray crystallography using MAD. Organic Letters 6, 1099-1102 (2004).
19. Adams, P. L. et al. Crystal structure of a group I intron splicing intermediate. Rna—a Publication of the Rna Society 10, 1867-1887 (2004).
20. Carrasco, N., Buzin, Y., Tyson, E., Halpert, E. & Huang, Z. Selenium derivatization and crystallization of DNA and RNA oligonucleotides for X-ray crystallography using multiple anomalous dispersion. Nucleic Acids Research 32, 1638-1646 (2004).
21. Huang, Z., Carrasco, N. & Du, Q. Systematic nucleotide oxygen substitution with selenium for structure determination using X-ray crystallography and NMR. Abstracts of Papers of the American Chemical Society 226, 699-ORGN (2003).
22. Serganov, A. et al. Structural basis for Diels-Alder ribozyme-catalyzed carbon-carbon bond formation. Nature Structural & Molecular Biology 12, 218-224 (2005).
23. Czyzewskachlebny, J. & Michalska, M. Tellurocarbohydrates. Journal of Chemical Society—Chemical Communications, 693-694 (1985).
24. Wu, W. Z. at al. Desulfurization of flue gas: SO2 absorption by an ionic liquid. Angewandte Chemie—International Edition 43, 2415-2417 (2004).
25. Huang, C. P., Chen, B. H., Zhang, J., Liu, Z. C. & Li, Y. X. Desulfurization of gasoline by extraction with new ionic liquids. Energy & Fuels 18, 1862-1864 (2004).
26. Paoli, M. L., Piccini, S., Rodriquez, M. & Sega, A. Sensible improvements induced by ionic liquids in the reaction of modified carbasugars with bases for the building of constrained carbanucleosides. Journal of Organic Chemistry 69, 2881-2883 (2004).
27. Uzagare, M. C., Sanghvi, Y. S. & Salunkhe, M. M. Application of ionic liquid 1-methoxyethyl-3-methyl imidazolium methanesulfonate in nucleoside chemistry. Green Chemistry 5, 370-372 (2003).
28. Khalafi-Nezhad, A. & Mokhtari, B. Tetrabutylammonium bromide: an efficient media for dimethoxytritylation of the 5'-hydroxyl function of nucleosides. Tetrahedron Letters 45, 6737-6739 (2004).
29. N. D. P. Cosford, R. F. Schinazi—J. Org. Chem., 1991, 56, 2161
30. M. J. Nemer, K. K. Ogilvie—Tetrahedron Lett., 1980, 21, 4149
31. K. Mori, C. Boiziau, C. Cazenave, M. Matsukura, C. Subasinghe, J. S. Cohen, S. Broder, J. J. Toulme, C. A. Stein—Nucleic Acids Res., 1989, 17, 8207
32. W. J. Stec, G. Zon, W. Egan, B. Stec—J. Am. Chem. Soc., 1984, 106, 6077
33. J. Stawinski, M. Thelin—Tetrahedron Lett., 1992, 33, 7255
34. J. Stawinski, M. Thelin—J. Org. Chem., 1994, 59, 130

1. A process for carrying out a reaction between a Group 16 element and an organic or inorganic compound, said Group 16 element being in elemental form, characterised in that the reaction medium in which the reaction takes place comprises at least one ionic liquid, with the proviso that the Group 16 element is not oxygen.

2. A process according to claim 1, wherein the reaction medium comprises, in admixture, (a) an ionic liquid and (b) sulfur, selenium or tellurium.

3. A process according to claim 2 where the reaction medium comprises in admixture an ionic liquid and sulfur.

4. A process according to claim 1 wherein at least a portion of the group 16 element is in solution in the ionic liquid.

5. A process according to claim 4 wherein the concentration of the group 16 element in solution is at least 0.05 g g⁻¹ when measured at 110° C.

6. A process according to claim 4 wherein the concentration of the group 16 element in solution is at least 0.10 g g⁻¹ when measured at 110° C.

7. A process according to claim 4 wherein the concentration of the group 16 element in solution is at least 0.20 g g⁻¹ when measured at 110° C.

8. A process according to claim 4 wherein the concentration of the group 16 element in solution is at least 0.40 g g⁻¹ when measured at 110° C.

9. A process according to claim 4 wherein the concentration of the group 16 element in solution is at least 0.70 g g⁻¹ when measured at 110° C.

10. A process according to claim 1, wherein the ionic liquid anion is a halide.

11. A process according to claim 1 wherein, the ionic liquid comprises at least one soft anion.

12. A process according to claim 11, wherein the soft anion is aromatic.

13. A process according to claim 12, wherein the soft anion is basic.

14. A process according to claim 11, wherein the soft anion may be selected from: [S₂CNR₂]⁻, [S₂CSR]⁻, [S₂COR]⁻ and [S₂CNR₂]⁻, wherein R may be hydrogen, a C₁ to C₄₀ straight chain or branched alkyl group, a C₃ to C₈ cycloalkyl group, or a C₅ to C₁₀ aryl group, and wherein said alkyl, cycloalkyl or aryl groups may be unsubstituted, or substituted by one to three groups selected from: C₁ to C₆ alkoxy, C₆ to C₁₀ aryl, CN, OH, NO₂, C₇ to C₃₀ aralkyl or C₇ to C₃₀ alkaryl.

15. A process according to claim 14, wherein R is selected from a C₁ to C₁₀ straight chain or branched alkyl group, a C₅ to C₇ cycloalkyl group, or a C₅ to C₈ aryl group, wherein said alkyl, cycloalkyl or aryl groups are unsubstituted or may be substituted by one to three groups selected from; C₁ to C₆ alkoxy, C₆ to C₈ aryl, CN, OH, NO₂, C₈ to C₁₅ aralkyl or C₈ to C₁₅ alkaryl.

16. A process according to claim 15, wherein R is selected from a C₁ to C₆ straight chain or branched alkyl group a C₅ to C₆ cycloalkyl group, or a C₅ to C₆ aryl group, wherein said alkyl, cycloalkyl or aryl groups are unsubstituted or may be substituted by one to three groups selected from: C₁ to C₆ alkoxy, C₆ to C₈ aryl, CN, OH, NO₂, C₈ to C₁₅ aralkyl or C₈ to C₁₅ alkaryl.

17. A process according to claim 11, wherein the soft anion is selected from: [O₂CR]⁻ wherein R is a C₁ to C₄₀ straight chain or branched alkyl group substituted by one to three OH groups.

18. A process according to claim 17, wherein R is a C₁ to C₆ straight chain alkyl group substituted by one OH group.

19. A process according to claim 18, wherein R is —CH(OH)CH₃.

20. A process according to claim 1 wherein the ionic liquid comprises an anion selected from: [S₂CSBu]⁻, [(S₂CCH₂CH₂)S]²⁻, [(S₂CSCH₂)]²⁻, [S₂CNEt₂]⁻, [S₂CN(CHMe₂)₂]⁻, [SO₃(CH₂)₂SH]⁻, [S₂CN(CH₂)₂O(CH₂)₂]⁻, [S₂CSN(CH₂)₄]⁻, [S₂COMe]⁻, [S₂COEt]⁻, [S₂COCHMe₂]⁻, [S₂COBu]⁻ and [S₂COPent]⁻.

21. A process according to claim 1 wherein the ionic liquid is an imidazolium, pyridinium, pyridazinium, pyrazinium, oxazolium, triazolium, pyrazolium, pyrrolidinium, piperidinium, tetraalkylammonium or tetraalkylphosphonium salt.

22. A process according to claim 21 imidazolium, pyridinium, pyridazinium, pyrazinium, oxazolium, triazolium, pyrazolium, pyrrolidinium, piperidinium, tetraalkylammonium or tetraalkylphosphonium halide salt.

23. A process according to claim 1 wherein the ionic liquid comprises an imidazolium halide salt.

24. A process according to claim 1 wherein the ionic liquid is selected from a compound of formula: 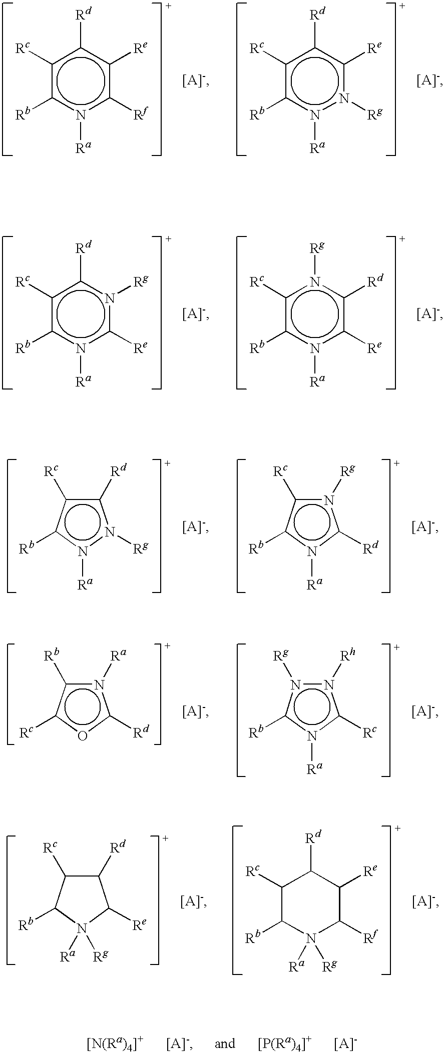
wherein
each R^{a} is independently selected from a C₁ to C₄₀ linear or branched alkyl or a C₃ to C₈ cycloalkyl group, wherein said alkyl or cycloalkyl group which may be substituted by one to three groups selected from: C1 to C₆ alkoxy, C₆ to C₁₀ aryl, C₁ to C₃₀ aralkyl and C₁ to C₃₀ alkaryl;
each R^{b}, R^{c}, R^{d}, R^{e}, R^{f}, R^{g} and R^{h} can be the same or different and are each independently selected from H or any of the R^{a} groups as defined above; and
[A]⁻ represents a halide anion.

25. A process according to claim 1 wherein the ionic liquid is selected from a compound of formula: 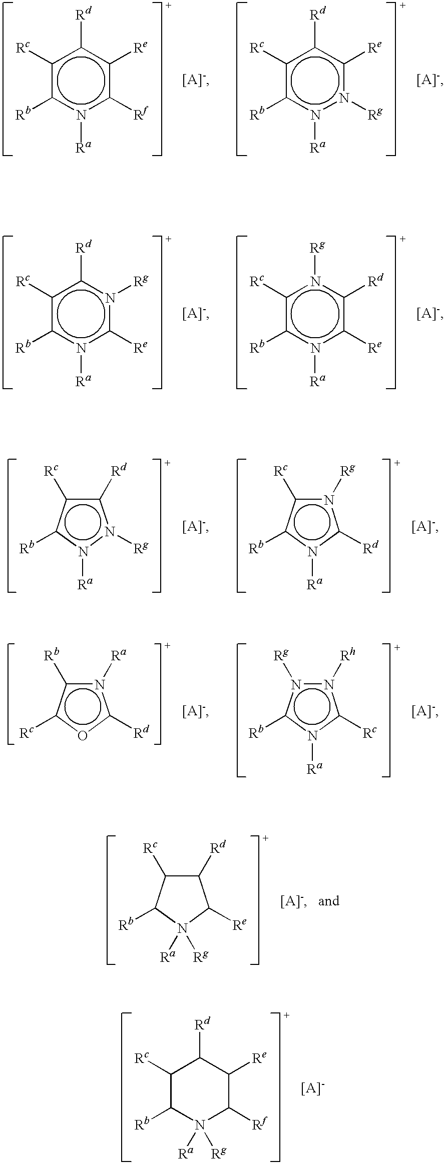
wherein R^{a}-R^{h} and [A]⁻ are as defined in claim 24.

26. A process according to claim 1 wherein the ionic liquid is selected from a compound of formula: 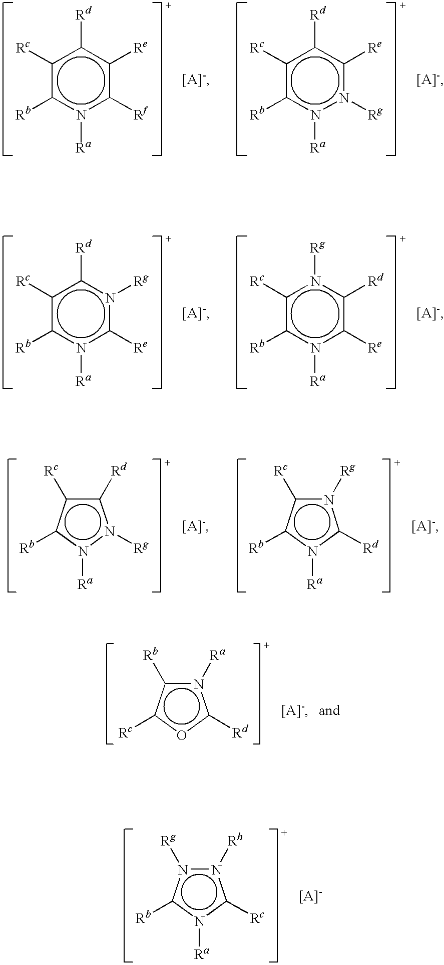
wherein R^{a}-R^{h} and [A]⁻ are as defined in claim 24.

27. A process according to claim 1 wherein the ionic liquid is selected from a compound of formula: 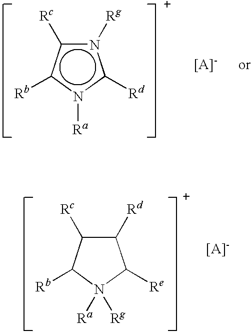
wherein [A]⁻, R^{a}, R^{b}, R^{c}, R^{d}, R^{e} and R^{g} are as defined in claim 4.

28. A process according to claim 1 wherein the ionic liquid is selected from a compound of formula: 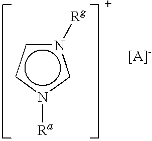
wherein R^{a}, R^{g} and [A]⁻ are as defined in claim 24.

29. A process according to claim 24 wherein each R^{a} represents C₁ to C₄₀ linear or branched alkyl.

30. A process according to claim 24 wherein each R^{g} and R^{h} represents C₁ to C₄₀ linear or branched alkyl.

31. A process according to claim 24 wherein R^{b}, R^{c}, R^{d}, R^{e}, R^{f}, R^{g} and R^{h} each represents hydrogen.

32. A process according to claim 24 wherein R^{a}, R^{g} and R^{h} each represents a C₁-C₂₀ alkyl group.

33. A process according to claim 24 wherein [A]⁻ represents Cl⁻ or Br⁻.

34. A process according to claim 24 wherein [A]⁻ represents Cl⁻.

35. A process according to claim 24 wherein the ionic liquid is selected from 1,3-ethylmethylimidazolium chloride or 1,3-butylmethylimidazolium chloride.

36. A process according to claim 1, wherein the organic compound is a phosphine.

37. A process according to claim 36 wherein the phosphine has the formula: P(R^{p})₃,
wherein each R^{p} group (which may be the same or different) is independently selected from a C₁ to C₁₀ linear or branched alkyl or a C₃ to C₈ cycloalkyl group, C₆ to C₁₀ aryl, C₁ to C₁₅ aralkyl and C₁ to C₁₅ alkaryl; and wherein said alkyl, cycloalkyl, aryl, alkaryl and aralkyl groups may be unsubstituted or substituted by one to three groups selected from nitro, halo, C₁ to C₆ alkoxy, oxo or hydroxyl groups.

38. A process according to claim 37 wherein each R^{p} group represents an unsubstituted or substituted aryl group

39. A process according to claim 38 wherein each R^{p} group represents phenyl.

40. A process according to claim 1 wherein the organic compound is a phosphate ester.

41. A process according to claim 1 wherein the organic compound is a nucleoside, nucleotide, or nucleoside or nucleotide derivative.

42. A process according to claim 1 wherein the nucleoside derivative is a nucleoside H-phosphonate

43. A process according to claim 41 wherein the organic compound is an H-phosphonates represented by the general formula:

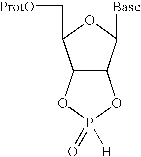

44. A process according to claim 41 wherein the organic compound is an internucleoside phosphite ester represented by the general formula: 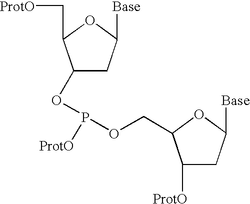
wherein each of the entities “Base” and “Prot” (which may be the same or different) are as defined above,

45. A process according to claim 41 wherein the solid support may be either solid phase or liquid phase.

46. A process according to claim 45 wherein the organic compound is a solid-support linked internucleoside phosphite esters represented by the general formula: 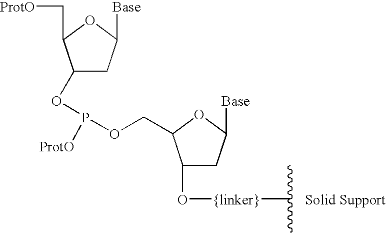
wherein each of the entities “Base” and “Prot” (which may be the same or different) are as defined above,

47. A reaction medium comprising an organic ionic liquid and at least one Group 16 element other than oxygen, said Group 16 element being in elemental form.

48. A reaction medium according to claim 47 wherein the Group 16 element is selected from sulfur, selenium and tellurium.

49. A reaction medium according to claim 47 wherein the Group 16 element is sulfur.

50. A reaction medium according to claim 47 wherein the ionic liquid comprising an anion which is a halide or a soft anion, and the cation is selected from imidazolium, pyridinium, pyridazinium, pyrazinium, oxazolium, triazolium, pyrazolium, pyrrolidinium, piperidinium, tetraalkylammonium or tetraalkylphosphonium salt.

51. A process for the production of a reaction medium according to claim 46, said process comprising admixing an organic ionic liquid with a Group 16 element other than oxygen.

52. A process according to claim 51 wherein the admixing is conducted at or beyond the melting point of the organic ionic liquid.

53. A process according to claim 50 wherein the Group 16 element is selected from sulfur, selenium and tellurium.

54. A process according to claim 51 wherein the ionic liquid is comprising an anion which is a halide or a soft anion, and the cation is selected from imidazolium, pyridinium, pyridazinium, pyrazinium, oxazolium, triazolium, pyrazolium, pyrrolidinium, piperidinium, tetraalkylammonium or tetraalkylphosphonium salt.

55. Use of an ionic liquid as a reaction medium for carrying out a sulfation reaction in which elemental sulfur is used as a sulfurisation agent.

56. Use of an ionic liquid as a reaction medium for carrying out a telluration reaction in which elemental tellurium is used as a tellurisation agent.

57. Use of an ionic liquid as a reaction medium for carrying out a selenation reaction in which elemental selenation is used as a selenisation agent.
